(19) United States
(12) Plant Patent Application Publication
(10) Pub. No.: US 2007/0079411 P1
(43) Pub. Date: Apr. 5, 2007

(54) Astilbe plant named 'Key West'

(76) Inventor: Hendricus M.J. Holtmaat, Zuidwolde (NL)

Correspondence Address: BIOLOGICAL PATENT SERVICES, LLC, 8424 40th AVE N., NEW HOPE, MN, 55427, US

(21) Appl. No.: 11/633,754

(22) Filed: Dec. 5, 2006

(30) Foreign Application Priority Data
Nov. 28, 2003 (NL) ABE 25

(51) Int. Cl. A01H 5/00

(52) U.S. Cl. PLT/263

(57) ABSTRACT

A new cultivar of Astilbe named ‘Key West’, characterized by leaves that emerges red and mature to a bronze-green with a red margin, abundant flowers that open red and change to pink on red flower stems, a long blooming habit with flowers present from late July until early September in the Northwest region of the U.S., and hardiness in U.S.D.A. Zones 3 to 9.

### BOTANICAL CLASSIFICATION

Astilbe hybrid

### VARIETAL DENOMINATION

‘Key West’

### BACKGROUND OF THE INVENTION

The present invention relates to a new and distinct cultivar of Astilbe hybrid in the Simplicifolia Group of hybrids and will be referred to hereafter by its cultivar name, ‘Key West’. ‘Key West’ represents a new Astilbe, an herbaceous perennial grown for landscape use.

‘Key West’ was selected by the inventor in summer of 2000 from a controlled breeding program in Zuidwolde, The Netherlands. ‘Key West’ was selected from a cross made between the seed parent Astilbe ‘Sprite’ (not patented), an Astilbe hybrid in the Simplicifolia Group of hybrids and the pollen parent Astilbe ‘Fanal’ (not patented), an Astilbe×arendsii hybrid.

Asexual reproduction of the new cultivar was first accomplished by in vitro propagation in Enkhuizen, The Netherlands under the direction of the inventor in 2000. It has been determined by propagation both by division and tissue culture that the characteristics of the new cultivar are stable and are reproduced true to type in successive generations.

### SUMMARY OF THE INVENTION

The following traits have been repeatedly observed and represent the characteristics of the new cultivar after observing plants grown outdoors in a field for two years in Mossyrock, Wash. These attributes in combination distinguish ‘Key West’ as a unique cultivar of Astilbe.
1. ‘Key West’ exhibits leaves that emerged red and gradually turn to a bronze/green color with red margins.
2. ‘Key West’ blooms heavily with 4 to 5 flower stems per 2 to 3 eye division.
3. The flowers of ‘Key West’ emerge red and fade to pink.
4. The flower stems of ‘Key West’ are primarily red in color.
5. ‘Key West’ has a long blooming habit, blooming for about 6 weeks from late July to early September in the upper Northwest section of the U.S.
6. ‘Key West’ obtains a height of 30 to 35.5 cm (12 to 14 inches) and a spread of 38 cm (15 inches).
7. ‘Key West’ is suitable for gardens and for container growing.
8. ‘Key West’ is hardy in U.S.D.A. Zones 3 to 9.

‘Key West’ differs from the seed parent, ‘Sprite’, in that it is longer blooming, has darker, more bronze foliage, flowers about a week earlier, and has flowers that emerge red before turning pink; the flowers of ‘Sprite’ are pink throughout development.

‘Key West’ differs from the pollen parent, ‘Fanal’, in that ‘Key West’ is longer blooming and retains a bronze cast to the foliage whereas the foliage of ‘Fanal’ emerges red and turns green. The flowers of ‘Fanal’ are dark red in color and do not turn pink like the flowers of ‘Key West’.

In comparison to ‘Key Largo’ (U.S. plant patent pending), ‘Key West’ is slightly more compact, has flowering stems that are more red, and leaves that are red and bronze-green rather than green. The flowers of ‘Key Largo’ are pink through out development.

### BRIEF DESCRIPTION OF THE PHOTOGRAPHS

The accompanying colored photographs illustrate the overall appearance and distinct characteristics of the new Astilbe. The plants in the photographs were grown for one year from a 2 to 3 eye division.

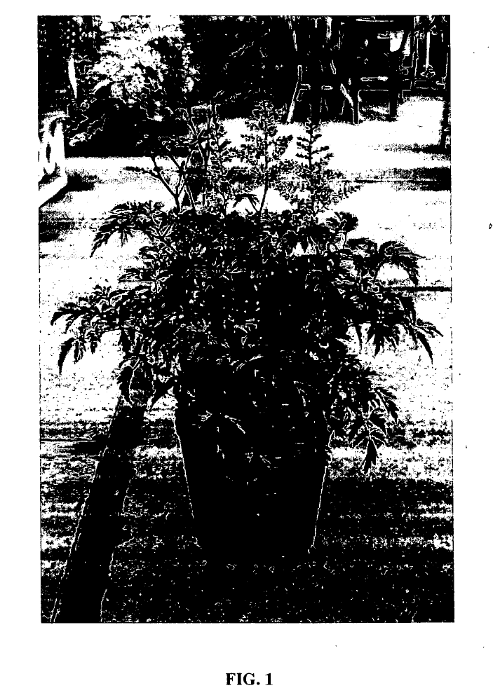

The photograph in FIG. 1 provides an overall view of a plant of ‘Key West’ as grown outdoors in a one-gallon container.

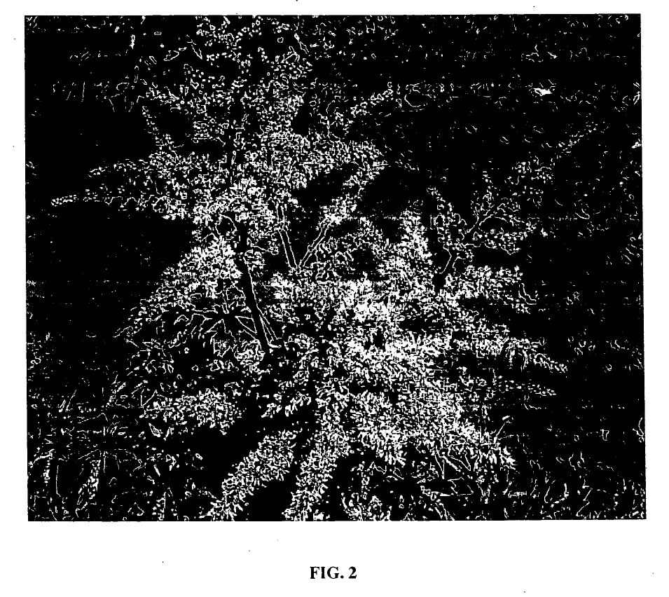

The photograph in FIG. 2 is a close-up view of the flowers of ‘Key West’ on a plant that was field grown. The colors in the photographs are as close as possible with the photographic and printing technology utilized. The color values cited in the detailed botanical description accurately describe the colors of the new Astilbe.

### DETAILED BOTANICAL DESCRIPTION

The following is a detailed description of the new cultivar as grown outdoors in one gallon containers in Mossyrock, Wash. The phenotype of the new cultivar may vary with variations in environmental, climatic, and cultural conditions, as it has not been tested under all possible environmental conditions. The color determination is in accordance with the 2001 RHS Colour Chart of The Royal Horticultural Society, London, England, except where general color terms of ordinary dictionary significance are used.
Botanical classification: ‘Key West’ is a cultivar of hybrid Astilbe in the Simplicifolia Group of hybrids.
General description:
   Blooming period.—About 6 weeks from late July into early September.
   Plant type.—Herbaceous perennial.
   Plant habit.—Clump-forming, upright flowering stems with cascading foliage, relatively compact.
   Height and spread.—Reaches 30 to 35.5 cm (12 to 14 inches) in height and about 38 cm (15 inches) in width.
   Hardiness.—U.S.D.A. Zone 3 to 9.
   Culture.—Prefers humus-rich, moist but well-drained soils in partial shade or full sun in areas where summers are cool with sufficient moisture.
   Diseases and Pests.—No unique susceptibility or resistance to diseases observed, Astilbe is typically disease free.
   Root description.—Fibrous, emerging from rhizome.
Growth and propagation:
   Propagation.—Rhizome Division and tissue culture.
   Growth rate.—Moderate.
   Cropping time to bloom.—A bare root 2 to 3 eye division will finish in a one-gallon container in about 90 days when grown outdoors with an average temperature of 12° C. under ambient light.
Stem description:
   Stem shape.—Slightly ovoid, swollen at nodes and base of petiole.
   Stem color.—Young and mature stems 145A, heavily suffused with 183A.
   Stem size.—Up to 25 cm in length and up to 4 cm in diameter.
   Stem surface.—Glabrous.
   Stem aspect.—Strong, mostly held upright.
   Branching habit.—Basal branching only.
Foliage description:
   Leaf division.—Bi-ternate.
   Leaf attachment.—Petiolate.
   Leaf arrangement.—Alternate.
   Leaf aspect.—Held nearly horizontal with leaflets concave to mid rib.
   Leaf size.—Average of 9 cm in length and 8 cm in width.
   Leaflet shape.—Terminals and laterals; ovate to broadly lanceolate.
   Leaflet base.—Cuneate.
   Leaflet apex.—Attenuate.
   Leaflet venation.—Pinnate, not conspicuous, color is the same as the leaf color on upper and lower surface.
   Leaflet margins.—Biserrate with irregular to maple leaf-like deep incisions.
   Leaflet size.—Lateral leaflets; up to 4 cm in length and 3 cm in width, terminal leaflets; an average of 6 cm in length and 6 cm in width, both include secondary leaflets.
   Leaflet surface.—Upper surface; glabrous with sheen, lower surface; very sparsely pubescent.
   Leaflet color.—New foliage upper and lower surface; 138A heavily suffused with 187A, mature foliage upper surface; N189A with very edge of margin 187A, mature foliage lower surface; 137B with some overlay of 187A.
   Petioles.—Color is 144A and suffused with 178A, ranges 2 to 4 mm in diameter and ranges from about 3 to 8 cm in length, surface is glabrous to sparsely pubescent.
   Petiolules.—Color is 144A and suffused with 178A to varying degrees when mature, new foliage color is N144A, about 1 mm in width and 2 to 4 cm in length for the primary petiolules and 0.5 to 2 cm in length for the secondary petiolules, surface is glabrous.
Flower description:
   Inflorescence type.—Numerous rotate single flowers arranged on pyramidal panicles and single racemes emerging from the base of the peduncles.
   Inflorescence size.—Reaches up to 15 cm in height and about 6 cm in width in full bloom.
   Flower fragrance.—None detected.
   Flower quantity.—Average of 10 flowering stems per two gallon sized plant, up to 1000 flowers per flowering stem.
   Flower lastingness.—Average of 10 days per flower, individual panicles blooms for about 3 weeks, flowers persistent.
   Flower buds.—Globose in shape, average of 2 mm in diameter and 3 mm in length, 145D in color suffused with 71A on upper portion of sepals.
   Flower aspect.—Held at about a 45° angle from Rachis.
   Flower shape.—Bell shaped becoming more flared.
   Flower size.—About 4 mm in depth and 6 mm in diameter.
   Petals.—About 5, linear to lanceolate to oblanceolate in shape and emerge from between sepals and held at about a 45° angle, margin is entire, apex is acute, upper and lower surface is glabrous, color of upper and lower surface is 71A to 71B changing to 73A to 73B as they mature, about 4 mm in length and 0.7 mm in width.
   Calyx.—Campanulate, sepals fused at base.
   Sepals.—5, elliptic in shape, margin is entire, apex is acute, base is fused, surface is smooth in appearance, color of upper and lower surface is 160C and heavily suffused with 71A, about 1 mm in width and 2 mm in length.
   Bracts.—2, lanceolate in shape, 160C in color suffused with 71A, about 0.5 mm in width and 2 mm in length, acute apex, base fused to rachis.
   Peduncles.—About 3 to 6.5 cm in length and an average of 2 mm in width, held nearly upright, strong, color 178A.
   Pedicels.—About 0.5 mm in length and 0.5 mm in width, 160C in color suffused with 71A, pilose surface.
   Rachis.—About 13 cm in length, with secondary branches up to 5 mm in length, up to 2 mm in width with secondary branches about 1 mm in width, maturing to N79A in color, surface is pilose with hairs mostly N79A in color.
Reproductive organs:
   Gynoecium.—3 Pistils, club-shaped, about 2 mm in length and 1.5 mm in width, stigma 59A in color and minute, style is continuous with superior ovary and is about 2 mm in length and 1.5 mm in width at base, 71 to 71B in color.
   Androcoecium.—About 14 stamens, anthers are oblong but stout in shape, basifixed, about 0.2 mm in length and width and N187A in color, filaments are about 2 mm in length and 71C in color, pollen is not visible.
   Seed.—Seed development has not been observed.

I claim:

1. A new and distinct cultivar of Astilbe plant named ‘Key West’ as herein illustrated and described.
